(19) United States
(12) Patent Application Publication
(10) Pub. No.: US 2019/0307333 A1
(43) Pub. Date: Oct. 10, 2019

(54) ACOUSTOELECTRIC IMAGE-GUIDED THERAPY

(71) Applicant: ARIZONA BOARD OF REGENTS ON BEHALF OF THE UNIVERSITY OF ARIZONA, Tucson, AZ (US)

(72) Inventors: Russell WITTE, Tucson, AZ (US); Charles M. INGRAM, Tucson, AZ (US); Qian LI, Tucson, AZ (US); Yexian QIN, Tucson, AZ (US)

(73) Assignee: Arizona Board of Regents on Behalf of the University of Arizona, Tucson, AZ (US)

(21) Appl. No.: 16/306,664

(22) PCT Filed: Jun. 5, 2017
(86) PCT No.: PCT/US2017/035963

§ 371 Date: Dec. 3, 2018

Related U.S. Application Data
(60) Provisional application No. 62/345,377, filed on Jun. 3, 2016.

(51) Int. Cl. A61B 5/00; A61B 18/02; A61B 18/08; A61B 18/12; A61N 7/02

(52) U.S. Cl. CPC A61B 5/0093; A61B 5/4836; A61B 18/02; A61B 2017/320069; A61B 18/12; A61N 7/02; A61B 18/08

(57) ABSTRACT

An acoustoelectric image-guided therapy system and method for applying acoustoelectric image-guided therapy are disclosed. Embodiments are directed to therapy systems and methods that utilize and are based on acoustoelectric imaging. The acoustoelectric imaging may be combined with focused therapies such as ultrasound, ablation, or hyperthermia for treatment of, for example, acute or chronic conduction abnormalities (e.g., neuropathy, arrhythmia, epilepsy, etc.). Current-density maps are obtained near the region of interest using acoustoelectric imaging techniques. Targeting of the region of interest is then identified for therapy based on the current-density maps. Treatment of the targeted region of interest is then employed while optionally monitoring bioelectric properties using real-time feedback from the acoustoelectric imaging. Imaging and therapy may be automatically co-registered. Standard electrophysiology (e.g., ENG, ECG, EEG, etc.) may be performed simultaneously with the same electrodes used for the acoustoelectric imaging.

### CROSS REFERENCE TO RELATED APPLICATION(S)

This application claims priority to U.S. provisional patent application No. 62/345,377, filed on Jun. 3, 2016, which is hereby incorporated herein by reference in its entirety.

### GOVERNMENT SPONSORSHIP

This invention was made with government support under Grant No. R01 EB009353 awarded by NIH. The government has certain rights in the invention.

### FIELD OF THE INVENTION

Embodiments are in the field of therapy systems and methods for applying therapy. More particularly, embodiments disclosed herein relate to therapy systems and methods that utilize and are based on acoustoelectric imaging.

### BACKGROUND OF THE INVENTION

Imaging techniques such as those described in U.S. Pat. No. 8,057,390 issued to Witte et al. on Nov. 15, 2011 are known. A current source density mapping system, such as that described in the '390 patent, includes an ultrasound transducer emitting an ultrasound wave traveling along an ultrasound beam directed at a mapping field in a region of living tissue and an ultrasound pulser delivering a transmit pulse to the ultrasound transducer. The system includes a timing device producing controlled excitation of the transmit pulse; a plurality of recording electrodes positioned in contact with the living tissue detecting an acoustoelectric voltage signal generated at a bioelectric current source and within a focal zone of said ultrasound beam. An amplifier operatively connected to the recording electrodes amplifying the acoustoelectric voltage signal at a predetermined gain; and an analyzing component comprising a digitizer, a sampling device, a signal processor and a display unit operatively connected to the amplifier determining the location of the bioelectric current source by analyzing the acoustoelectric voltage signal detected by the recording electrodes in response to an interaction between the ultrasound wave and the presence of a current source in the mapping field.

Current therapy techniques are somewhat deficient in efficacy and accuracy and can lack sufficient spatial resolution. In addition, the procedure time is increased due to the additional time required for mapping of the desired locations targeted for therapy, because the therapy mapping is separate and independent from the imaging mapping. An additional disadvantage of the separate and independent mapping is the additional devices and tools required for the therapy apparatus and equipment related thereto such as additional electrodes, etc. Additional calibration is also needed for the separate and independent imaging and therapy systems.

Thus, it is desirable to provide embodiments of a therapy system that utilize acoustoelectric imaging techniques (such as those described in the '390 patent) that do not suffer from the above drawbacks.

These and other advantages of the present invention will become more fully apparent from the detailed description of the invention hereinbelow.

### SUMMARY OF THE INVENTION

Embodiments are directed to an acoustoelectric image-guided therapy system. The system comprises: an acoustoelectric imaging system that generates a map of a region of interest; and a therapy system that targets the region of interest using the map, and that applies therapy to the targeted region of interest.

In an embodiment, the therapy system is an ultrasound therapy system.

In an embodiment, the therapy system is an ablation therapy system.

In an embodiment, the therapy system is selected from the group consisting of ultrasound ablation, radio frequency (RF) ablation, cryoablation, hyperthermia ablation, and a combination thereof.

In an embodiment, the therapy system is selected from the group consisting of transient opening of the blood brain barrier (BBB), sonoporation, ablation, hyperthermia, cavitation, necrosis, coagulation of blood vessels, sonothrombolysis, sonolysis, lithotripsy, neuromodulation, and a combination thereof.

In an embodiment, the acoustoelectric imaging system is selected from the group consisting of pulse-echo ultrasound, ultrasound current source density imaging (UCSDI), and a combination thereof.

In an embodiment, the therapy system is configured to use integrated feedback from the acoustoelectric imaging system while targeting the region of interest. The integrated feedback may be in real-time.

In an embodiment, the acoustoelectric imaging system comprises a transducer/probe used for generating the map, and wherein the therapy system is configured to target the region of interest using the transducer/probe.

In an embodiment, the acoustoelectric imaging system is configured to utilize passive electrical conduction to generate the map.

In an embodiment, the acoustoelectric imaging system is configured to utilize active electrical conduction to generate the map.

Additional embodiments and additional features of embodiments for the acoustoelectric image-guided therapy system and method for applying acoustoelectric image-guided therapy are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures. The detailed description will refer to the following drawings in which like numerals, where present, refer to like items.

FIG. 1 is a diagram illustrating the acoustoelectric effect.

FIGS. 2A and 2B are diagrams illustrating an embodiment of a set-up for UCSDI imaging and HIFU ablation.

FIGS. 3A and 3B are diagrams illustrating an embodiment of a demonstration of ultrasound current source density imaging (UCSDI)+HIFU in tissue samples. FIGS. 3A and 3B depict images of tissue ablation in preserved porcine tissue before and after HIFU (with a 3 MHz transducer/probe), respectively. As shown, R1 and R2 are the recording electrodes shown in FIG. 5B.

FIG. 4 is a diagram illustrating UCSDI images of tissue ablation in preserved porcine tissue before and after HIFU.

FIGS. 5A and 5B are diagrams illustrating an embodiment of a demonstration of UCSDI+HIFU in tissue samples. FIGS. 5A and 5B depict images of tissue ablation in fresh porcine tissue before and after HIFU (with a 3 MHz transducer), respectively.

FIG. 6 is a diagram illustrating UCSDI images of tissue ablation in fresh porcine tissue using HIFU.

FIG. 7 is a diagram depicting cross-sectional pulse-echo (PE) images for porcine tissue at ablation times corresponding to and automatically co-registered with UCSDI frames #1 and #6 in FIG. 6.

FIG. 8 is a diagram illustrating current (mA) and AE signal (μV) changes during HIFU ablation, using a comparison of two porcine samples.

FIGS. 9A and 9B are diagrams illustrating 3D UCSDI representations (volumes) of preserved porcine sample before and after ablation, respectively.

FIG. 10 is a diagram illustrating an embodiment of acoustoelectric cardiac imaging before, during, and after cryoablation.

FIG. 11 are diagrams depicting images of a rabbit heart with an electrode catheter on epicardium before/after cryoablation (at site indicated on right). Acoustoelectric images and ECG were acquired at each interval.

FIG. 12 is a diagram illustrating standard ECGs before and after cryoablation on 5 channels as indicated. Channel 4 (CH4) shows a dramatic change after cryoablation.

FIG. 13 is a diagram illustrating plots of standard ECG and acoustoelectric ECG at one position of US beam, before (darker lines) and after (lighter lines) cryoablation.

FIG. 14 is a diagram illustrating an AE image (XY slice, top row) and ECG (bottom row) before ablation (left column) and after ablation (right column).

FIG. 15 is a diagram illustrating an AE image (XY slice, top row) and ECG (bottom row) before ablation (left column) and after ablation (right column).

FIG. 16 is a diagram illustrating an AE B Mode color image (XZ) for Channel 4 only showing cross-section through right ventricle, before and after ablation.

FIG. 17 is a diagram illustrating an AE B Mode color image (XZ) for Channel 2 only showing cross-section through right ventricle, before and after ablation.

FIG. 18 is a flowchart illustrating an embodiment of a method for applying acoustoelectric image-guided therapy, in accordance with an embodiment.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical acoustoelectric/ultrasound imaging system (or method) or typical therapy system (or method). Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It's intended that all such additional devices, systems, methods, features, and advantages be protected by the accompanying claims.

With regard to imaging, the imaging involves a combination of ultrasound (i.e., a transmitted ultrasound beam) and at least one recording electrode. The transmitted beam generates an acoustoelectric interaction with tissue that changes the electrical properties of the tissue just transiently at the location of the ultrasound focus of the ultrasound beam. And that interaction can be considered essentially as a change in conductivity; i.e., it changes conductivity of the tissue at the specific location. When a current is flowing through the tissue it could be passive (i.e., passive electrical conduction), where one would inject a current, or active (i.e., active electrical conduction), where the natural currents in the body might come from the heart or the brain or the nerve or skeletal muscle that generates its own current. When this active current interacts with the ultrasound beam, there's an interaction signal that can be picked up on the recording electrode(s). And what's interesting about that signal is that it's an electrical measurement that is related to the local current source density of the tissue while the current is flowing through the tissue, but it's also confined to the ultrasound focus. Thus, an improved spatial resolution for imaging electrical activity or current flow in the body may be achieved, rather than from combining a number of electrodes and attempting to do a reconstruction. This technique is effectively an improvement of spatially confining an electrical measurement using the ultrasound beam.

The region that is imaged may be co-registered to the area that is applied therapy to (e.g., ablating). However, imaging data may alternatively be used at different times. For example, instead of using the same ultrasound beam during, for example, cryoablation, a stick with liquid nitrogen may be used to ablate some cardiac tissue, and then an imaging technique may be used to monitor the electrical activity that was an effect of the cryoablation. Thus, in the latter case, the same instrument is not actually used to do the therapy that was used for the imaging. Two different devices were used but the devices are still used together and possibly could be combined if used at the same time as well.

In embodiments, an imaging map may be used as feedback for the therapy/treatment itself, e.g., to determine when to stop treatment or perhaps whether the correct area of treatment is being treated. Thus, feedback in real-time is possible with the therapy.

In an embodiment where there are separate image and therapy devices, it may be necessary to avoid negative interaction between the devices. For example, when using an ultrasound transducer, an interweaving of pulses may be employed. In this example, a long pulse during therapy may be followed by downtime. Then, during the downtime, imaging could be performed between the therapeutic pulses, so there wouldn't necessarily be any significant interference between the two.

There are some other advantages as well but that's one of the main interesting aspects of this technique. So information that is obtained from the acoustoelectric imaging helps guide the therapy. As for the types of therapy, there could be lots of different possible applications of different types of therapies. Feedback from the acoustoelectric imaging is informative about the electrical properties of the tissue which could be inherent to the tissue itself like the heart or a nerve or it could be like an impedance imaging measurement where one is injecting current through the tissue. There are different scenarios where one could use this as feedback for monitoring the electrical properties of the tissue as the ablation of the tissue occurs or applying other therapy to the tissue, which would be one example of the therapy's ablation. So one can imagine there are scenarios where treating peripheral nerves (for example, neuropathic pain) and an ultrasound is one of the ways that can be employed to treat it; but it would be desirable to monitor the electrical conduction of the nerve while doing the ablation to get feedback on, for example, the times it stopped or some other signature one is looking for.

Embodiments are directed to an acoustoelectric image-guided therapy system which combines acoustoelectric-type ultrasound imaging with therapy which is co-registered based on the mapping from the ultrasound imaging. The acoustoelectric imaging that may be employed in any of the embodiments includes acoustoelectric impedance imaging and acoustoelectric current source density imaging (i.e., ultrasound current source density imaging (UCSDI)). However, other acoustoelectric imaging techniques may be contemplated.

Embodiments may perform acoustoelectric imaging of tissue electrical properties (impedance or current densities) during ablation therapy of the heart, brain, peripheral nerve or other types of tissue.

For example, in embodiments, UCSDI may be combined with focused therapies such as ultrasound, ablation, or hyperthermia for treatment of acute or chronic conduction abnormalities (e.g., neuropathy, arrhythmia, epilepsy, etc.). Current-density maps are obtained near the region of interest at the resolution of the ultrasound focus. Targeting of the region of interest is then identified for therapy (e.g., ablation, hyperthermia, etc.) based on the current density maps. Treatment of the targeted region of interest (e.g., using High-Intensity Focused Ultrasound (HIFU) pulses) is then employed to treat the targeted region of interest while optionally monitoring bioelectric properties in real-time. Imaging (e.g., standard pulse-echo ultrasound, UCSDI, etc.) and therapy (e.g., ultrasound, ablation, hyperthermia, cavitation, cryotherapy, etc.) are automatically co-registered. Standard electrophysiology (e.g., ENG, ECG, EEG, etc.) may be performed simultaneously on the same electrodes used for ultrasound imaging (e.g., UCSDI).

Different types of imaging may be applicable to embodiments described herein. Imaging applications that may be employed would cause tissue to be electrically excitable (e.g., the heart, the brain, muscle and nerves). In the heart, there's a broad application for ablation therapy where inter-cardiac mapping can be performed which is a routine procedure, wherein electrocardiac mapping may be performed prior to ablation for treatment of arrhythmia. Ills is one type of category where imaging/mapping is performed actually inside the heart; and then the ablation is most often done with radio frequency (RF) heating, e.g., with the electrode on the tip of a catheter. Other therapies, such as cryoablation, may similarly be contemplated when using focused ultrasound for that application. In the brain, focused ultrasound may be employed, such as through the skull and into the brain for treatment of brain tumors, Parkinson's, epilepsy, etc. Thus, for both the brain and the heart, there are a variety of different procedures being used including using ultrasound to treat these tissues. Embodiments described herein can also be extended for peripheral nerves and skeletal muscles. In these techniques, these target areas could be excited and their electrical activity may be observed with the acoustoelectric imaging technique. However, there may be other scenarios as well, such as by actually injecting current to perform, for example, impedance imaging. In this scenario, it's not required to have tissue that is itself electrically excitable. Rather, the current is being generated and the changes in the impedance of the tissue are being observed during the application of the generated current. That information may also be obtained from the acoustoelectric technique from injecting current through a piece of tissue and looking at the image as the ablation (or other type of therapy) occurs and one can observe the signal change as the ablation (or other type of therapy) occurs.

Imaging may be performed relatively deep within the body. The technique can go centimeters down and in some situations, depending on the frequency being used, one can go through the skull even and can resolve signals deep into the brain or into the body because the speed of sound is utilized for the depth information. Therefore, the acoustoelectric imaging technique being used is the same principal as typical ultrasound imaging. It has been shown that four-dimensional mapping may be achieved where volume is observed over time in the tissue. The ability to do this with an electrical signal is unique. As for the therapy aspect, the depth of application depends on the particular therapy, but if focused ultrasound therapy is employed, the focus target may be pinpointed down to a couple millimeters into the body.

Embodiments of the present disclosure utilize an application-specific apparatus, integrating various components, which is envisioned to facilitate adoption of the method and ease-of-use. The apparatus/system involves combining acoustoelectric imaging (i.e., UCSDI) hardware and software with HIFU therapy optionally using the same ultrasound transducer. The system also may use a custom chamber for imaging+therapy. Images of the tissue were taken before and after the procedure as mentioned with respect to the figures below.

The embodiments in this disclosure demonstrate that acoustoelectric imaging can provide feedback during ablation therapy (e.g., via ultrasound, RF, or cryoablation) or other types of hyperthermia.

Any of the embodiments described in this disclosure may provide for immediate and/or future applications of at least one or more of the following:

1) Provides bioelectrical feedback for peripheral nerve ablation, along with standard ultrasound imaging (all co-registered); peripheral neuropathy.
2) Provides bioelectrical feedback during HIFU treatment of cardiac arrhythmia treatment, along with standard ultrasound imaging (all co-registered).
3) Provides bioelectrical feedback during HIFU treatment of brain disorders (e.g., epilepsy, cancer, Parkinson's), along with standard ultrasound imaging (all co-registered).
4) Provides bioelectrical feedback during HIFU treatment of skeletal muscle disorders (e.g., myasthenia gravis, atrophy), along with standard ultrasound imaging (all co-registered).
5) Potentially competes with RF ablation (and cryoablation) treatment for cardiac arrhythmias by using image-guided HIFU ablation.
6) Potential targeting of non-sustained arrhythmias, like ventricular tachycardia (currently untreatable with existing mapping and ablation methods).
7) Potential for tracking bioelectric current during ultrasound therapy for the heart, brain, peripheral nerve, skeletal muscle, and other electrically-excitable tissue.

The embodiments described in this disclosure provide at least one or more of the following advantages:
Imaging is co-registered with therapy.
Improves ablation efficacy/accuracy; ablations guided by biocurrent density information relevant to the tissue being ablated.
Improved accuracy, improved spatial resolution, localization (reduced number of erroneous ablations).
Reduced procedure time spent on mapping compared to ElectroAnatomical Mapping (EAM). EAM procedures are typically used for ablation therapy in the heart and take a very long time. Electrodes are placed along the cardiac catheter, and then over 1 to 2 hours they will stimulate the heart and look at the contractions of the heart and move this electrode array around inside the heart to develop ElectroAnatomical maps that may be registered with the structure of the heart and the electrical conduction. This technique is used for detecting arrhythmias during this 1 to 2 hours period to find the location where they want to ablate the heart. Because the technique takes so long, the registrations are often not very good especially towards the end of the mapping. As a result, ablation of the heart must occur multiple times until the right spot is hit. Thus, this technique can be very imprecise. Because of the long procedure, one can generally only do this procedure for certain types of arrhythmias that are what are called sustained, meaning they can repeat themselves many times over a period of an hour or two hours. But there are certain types of arrhythmias that are not sustained. They may only happen for a minute or two minutes and they might go away which would require a real-time technique where one should be able to pick up those arrhythmias as well. Therefore, the acoustoelectric imaging technique may be a real-time technique of looking at the electrical conduction of the heart, and possibly in 3D, but also obtain the anatomical information because the echo ultrasound is present and is co-registered automatically to the electrical map.
Method requires a minimal # of electrodes for mapping current flow.
Imaging component is safe and does not require ionizing radiation.
Imaging system is potentially portable and real-time.

The biggest impact would be to utilize these techniques completely non-invasively which would mean having electrodes on the chest in order to then deliver ultrasound transthoracically and the signal can be picked up. An ECT signal may be utilized but it would have very little spatial resolution, so it would be desirable to use the ultrasound beam to get much better spatial information, perhaps to obtain a non-invasive way to diagnose an arrhythmia which is one possible application. Of course, an intracardiac technique could alternatively be employed where one would piggyback on an intracardiac catheter that already exists with its electrodes on it and so then the catheter would be configured for ultrasound imaging capability. Using just as few as two electrodes, multidimensional mapping of up to even four dimensional type of imaging may be contemplated. Using just two electrodes won't be optimum in terms of sensitivity, but would be useful as long as the associated smaller signal could be sufficiently detected. However, by strategically placing the electrodes and having as many electrodes as necessary to detect the signal, this would optimize the technique which would make the technique feasible. In theory, even one electrode and a ground may be contemplated and similarly feasible.

When dealing with cardiac tissue or the brain, the signal that is being observed is a very small signal with regards to physiologic current. There is a lot of other electrical activity in the brain or the heart and so the amplitude of the signal being measured is typically a lot smaller than that activity. But it's that activity that is actually being measured, and the way that this is accomplished is this interaction signal is occurring on a different timescale which is the ultrasound timescale. The ultrasound happens in a few microseconds. So, a megahertz pulse is generated over a few microseconds and the electrical activity in the brain is evolving on the milliseconds, tens of milliseconds, time scale, or a kilohertz signal. Since the interaction signal is on a megahertz scale, the way that that electrical signal that's coming from the brain is sampled, which is exactly what the inventors' are trying to measure, is that the ultrasound signal is pulsed. The ultrasound is pulsed every few hundred microseconds, for example. So every time it is pulsed, instantaneous information about the conduction is obtained, i.e., the electrical current in the brain at that moment in time. So if the ultrasound transducer is pulsed at a certain rate, for example five kilohertz, then the volume is sampled at five thousand times per second so one could then track in time the neural currents that are happening much lower than the ultrasound pulsing that is employed to make the images.

Although these systems described in the embodiments in this disclosure apply passive (or “artificial”) current (providing impedance information via the AE signal), the tracking of bioelectric current during ultrasound therapy using the same methods may alternatively be contemplated.

A key feature of the present disclosure is using image-guided integrated feedback. The imaging technique employed in the '390 patent generates imaging maps which can assist with the ablation in a separate operation, but it doesn't integrate therapy directly with the imaging or maps generated, either together or in real-time.

Embodiments are directed to an acoustoelectric image-guided therapy system comprising: an acoustoelectric imaging system that generates a map of a region of interest; and a therapy system that targets the region of interest using the map, and that applies therapy to the targeted region of interest.

In an embodiment, the therapy system is an ultrasound therapy system.

In an embodiment, the therapy system is an ablation therapy system.

In an embodiment, the therapy system is selected from the group consisting of ultrasound ablation, radio frequency (RF) ablation, cryoablation, hyperthermia ablation, and a combination thereof.

In an embodiment, the therapy system is selected from the group consisting of transient opening of the blood brain barrier (BBB), sonoporation, ablation, hyperthermia, cavitation, necrosis, coagulation of blood vessels, sonothrombolysis, sonolysis, lithotripsy, neuromodulation, and a combination thereof.

In an embodiment, the acoustoelectric imaging system is selected from the group consisting of pulse-echo ultrasound, UCSDI, and a combination thereof.

In an embodiment, the therapy system is configured to use integrated feedback from the acoustoelectric imaging system while targeting the region of interest. The integrated feedback may be in real-time.

In an embodiment, the acoustoelectric imaging system comprises a transducer/probe used for generating the map, and wherein the therapy system is configured to target the region of interest using the transducer/probe.

In an embodiment, the acoustoelectric imaging system is configured to utilize passive electrical conduction to generate the map.

In an embodiment, the acoustoelectric imaging system is configured to utilize active electrical conduction to generate the map.

Embodiments are also directed to a method for applying acoustoelectric image-guided therapy. FIG. 18 is a flowchart illustrating an embodiment of a method 1800 for applying acoustoelectric image-guided therapy, in accordance with an embodiment. In an embodiment, the method comprises: generating a map of a region of interest via an acoustoelectric imaging system (block 1802); and providing a therapy system for targeting the region of interest using the map, and for applying therapy to the targeted region of interest (block 1804).

In an embodiment, the therapy system is an ultrasound therapy system.

In an embodiment, the therapy system is an ablation therapy system.

In an embodiment, the therapy system is selected from the group consisting of ultrasound ablation, radio frequency (RF) ablation, cryoablation, hyperthermia ablation, and a combination thereof.

In an embodiment, the therapy system is selected from the group consisting of transient opening of the blood brain barrier (BBB), sonoporation, ablation, hyperthermia, cavitation, necrosis, coagulation of blood vessels, sonothrombolysis, sonolysis, lithotripsy, neuromodulation, and a combination thereof.

In an embodiment, the acoustoelectric imaging system is selected from the group consisting of pulse-echo ultrasound, UCSDI, and a combination thereof.

In an embodiment, the therapy system uses integrated feedback from the acoustoelectric imaging system while targeting the region of interest. The integrated feedback may be in real-time.

In an embodiment, the acoustoelectric imaging system comprises a transducer used for generating the map, and wherein the therapy system targets the region of interest using the transducer.

In an embodiment, the acoustoelectric imaging system utilizes passive electrical conduction for generating the map.

In an embodiment, the acoustoelectric imaging system utilizes active electrical conduction for generating the map.

Theory of Combined UCSDI and Ultrasound Therapy Technique

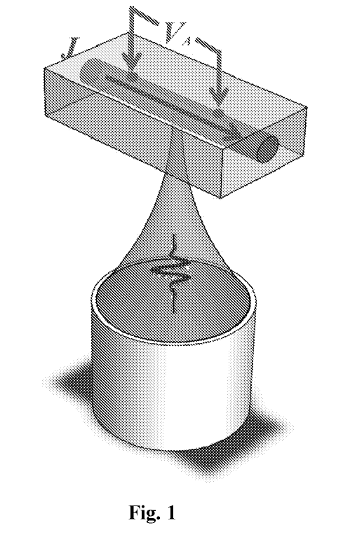

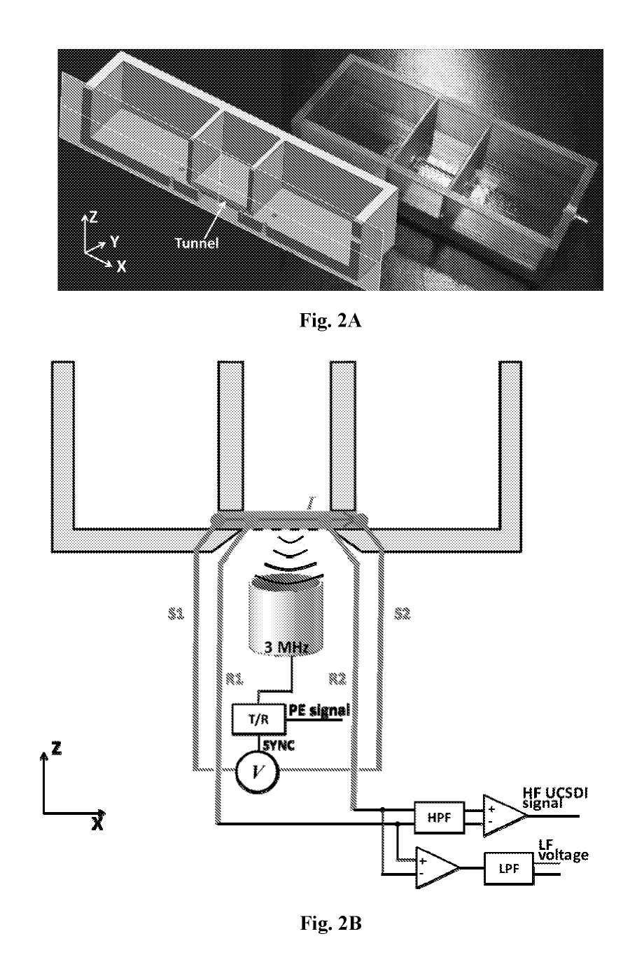

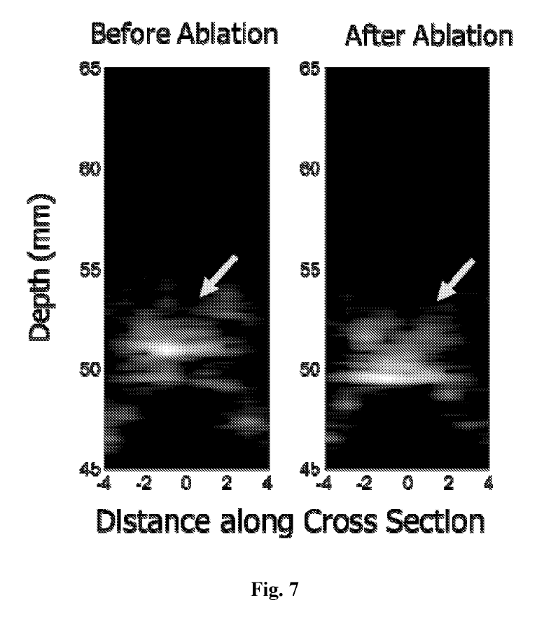

Bioelectrical current (J) passing through tissue induces a voltage drop (V) across the region of interest. When the tissue is pulsed with ultrasound, its resistance modulates along with the induced voltage. (FIG. 1) The magnitude of the voltage drop depends on the magnitude of the local pressure and current density and the acoustoelectric interaction constant, a material property of the tissue on the order of 0.1%/MPa. By scanning the sample with the ultrasound transducer, or using an array of transducers, a map is created of the sample's electrical properties (e.g., instantaneous current density distribution or resistivity), which may be related to the tissue's structure or physical properties, such as an infarct or scar tissue. This same transducer may simultaneously collect pulse-echo data for creating ultrasound images which are automatically co-registered with the map of current densities (FIG. 7). Pathological tissue or tissue targeted for therapy may then be treated (via ablation, acoustic cavitation or modest heating) (FIGS. 2A-5B). Since imaging and ablation may be done with the same transducer, high spatial accuracy is achieved. However, it is noted that an ultrasound probe that's been designed for therapy wouldn't necessarily be the same type designed for imaging. This may be due to, for example, differences in power requirements for imaging versus therapy, let alone the different types of therapies. In other words, in an example, an ultrasound transducer that's designed for (e.g., ablation) therapy is not typically optimized for imaging. That doesn't mean it can't be used for imaging, it's just not necessarily the ideal parameters one would choose for imaging something. So there may be some tradeoff in this situation in terms of using the same device for imaging and therapy. But in the case of cryoablation or RF ablation, then one would have a different device that's doing something to the tissue versus the device used for imaging. Feedback may still be employed, it's just that one may not have the precise control of where the ablation occurs versus where the imaging occurs. More image guidance would be needed rather than being automatically co-registered to the same location.

FIG. 1 is a diagram illustrating the acoustoelectric effect. Pressure from the ultrasound (US) pulse modulates resistance in the sample at the US frequency. This produces a momentary modulation in voltage (VAE) for a given bioelectric (or applied) current (J). The voltage modulation is a function of the pressure amplitude, beam size near the focus, local current density, acoustoelectric interaction constant of the tissue, and geometry. Volume images of the current densities (or impedances) may be obtained by scanning the US transducer across the region of interest.

FIGS. 2A and 2B are diagrams illustrating an embodiment of a set-up for UCSDI imaging and HIFU ablation. FIG. 2A shows a CAD design of an exemplary chamber, perspective X-Z cross-sectional view (left-side of figure) and an image (right-side of figure). The middle compartment contains a tunnel for placement of the samples. The two side compartments provide access for electrical coupling. FIG. 2B shows an instrumentation diagram for current injection, voltage modulation, and High-Intensity Focused Ultrasound (HIFU) ablation. T/R represents an ultrasound pulser/receiver, which is pulsed in synchrony with the function generator. HPF represents a high pass filter, LPF represents a low pass filter, LF represents low frequency, HF represents high frequency, S1 & S2 represent source electrodes, R1 & R2 represent recording electrodes. A function generator provides a low-frequency signal (e.g., 200 Hz sine or square wave), which is the source of the injected current. The high and low frequency electrical signals (i.e., the acoustoelectric (AE) signal and the original signal) are separated, amplified, and recorded by the back-end electronics. It is optional to replace the passive applied current with the intrinsic bioelectric current of the tissue (e.g., ECG or ENG).

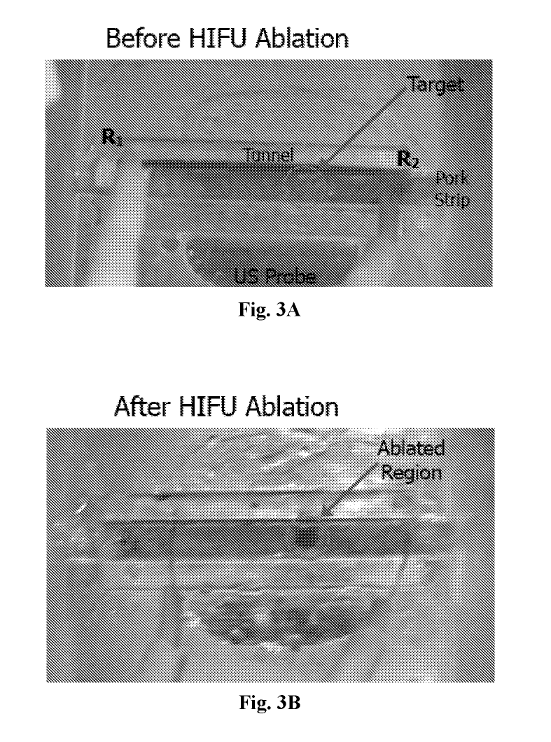

FIGS. 3A and 3B are diagrams illustrating an embodiment of a demonstration of UCSDI+HIFU in tissue samples. FIGS. 3A and 3B depict images of tissue ablation in preserved porcine tissue before and after HIFU (with a 3 MHz transducer), respectively. As shown, R1 and R2 are the recording electrodes shown in FIG. 5B.

This exemplary demonstration in this embodiment includes the following criteria:

Transducer: 3 MHz commercial HIFU ultrasound transducer;

Tissue: Preserved porcine tissue with 3×3 mm cross-sectional area;

Ablation: Twenty-four 10-20 second periods at ˜3 min intervals, using 3 MHz, continuous-wave (CW), with an intensity of 2.05 kW/cm² for each sequence;

Ultrasound: 3 MHz tone burst with 10 cycles, following each ablation period with pulse echo ultrasound & UCSDI (i.e., acoustoelectric imaging); and

Current source: Current is injected through the tissue by applying a 200 Hz square wave (10V peak-to-peak) across the sample.

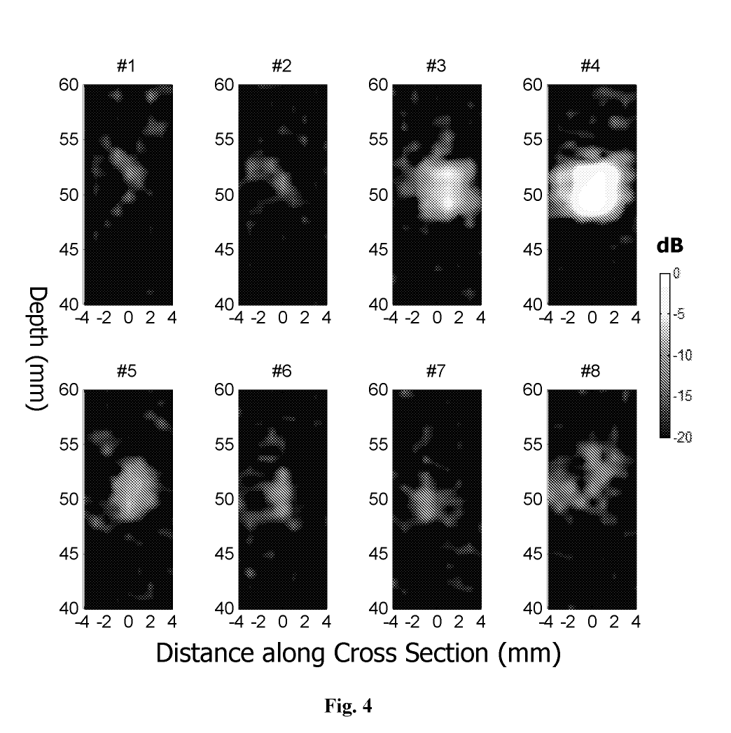

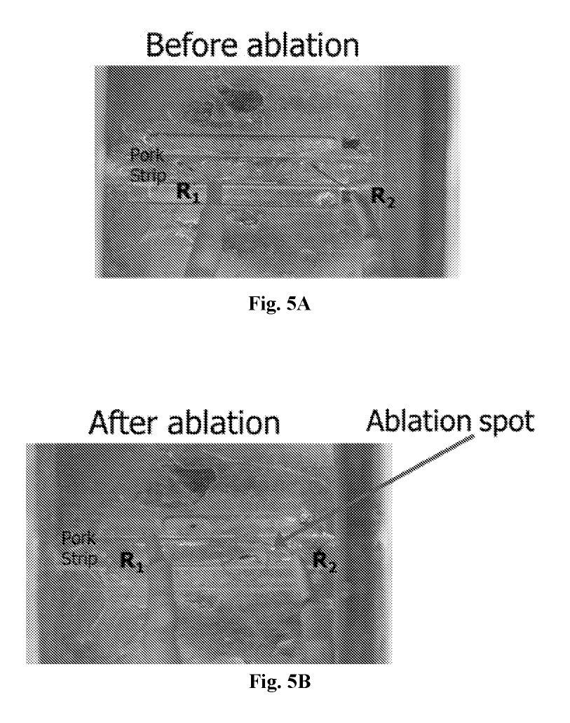

FIG. 4 is a diagram illustrating UCSDI images of tissue ablation in preserved porcine tissue before and after HIFU. The figure shows cross-sectional UCSDI images after successive ablation periods (˜15 sec, 3 MHz, 2.9 kW/cm²) for preserved porcine tissue. These images are a cross-section at the ablation spot shown in FIG. 3B. Note that the UCSDI signal increases with ablation at first, then decreases as tissue is destroyed. This indicates that the impedance initially dropped (consistent with a temperature rise) and then increased (consistent with cell destruction and thermal damage).

FIGS. 5A and 5B are diagrams illustrating an embodiment of a demonstration of UCSDI+HIFU in tissue samples. FIGS. 5A and 5B depict images of tissue ablation in fresh porcine tissue before and after HIFU (with a 3 MHz transducer), respectively. The image in FIG. 5B denotes an ablated region of fresh porcine tissue with HIFU using a 3 MHz HIFU transducer (˜15 sec, 0.73 kW/cm²) and driven at 1.5 MHz. Electrodes R₁ and R₂ record the AE voltage modulations from which the UCSDI image is created.

This exemplary demonstration in this embodiment includes the following criteria:

Transducer: 3 MHz HIFU ultrasound transducer;

Tissue: porcine strip with 3×3 mm cross-sectional area;

HIFU Ablation: Twelve 10-20 second periods at various intervals, using 3 MHz, CW, with intensity of 0.51 kW/cm² each time;

UCSDI: 1.5 MHz tone burst with 5 cycles, following each ablation period; and

Current source: 200 Hz square wave (10V peak-to-peak).

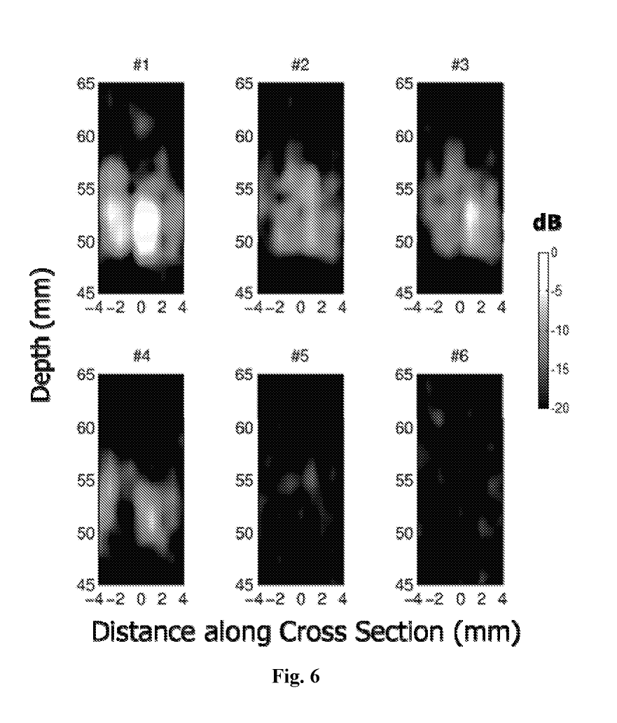

FIG. 6 is a diagram illustrating UCSDI images of tissue ablation in fresh porcine tissue using HIFU. The figure shows cross-sectional UCSDI images following successive ablation periods for fresh porcine tissue. The transducer is driven at 1.5 MHz, lowering the spatial resolution compared to the 3 MHz images of FIG. 4. These images in FIG. 6 are a cross-section of the ablation spot shown in FIG. 5B.

FIG. 7 is a diagram depicting cross-sectional pulse-echo (PE) images for porcine tissue at ablation times corresponding to and automatically co-registered with UCSDI frames #1 and #6 in FIG. 6. The arrows denote the tissue in the tunnel. While the PE images provide a structural frame of reference, it is unable to distinguish between the ablated and non-ablated tissue clearly differentiated by UCSDI in FIG. 6. Thus, standard US and UCSDI have different sources of contrast.

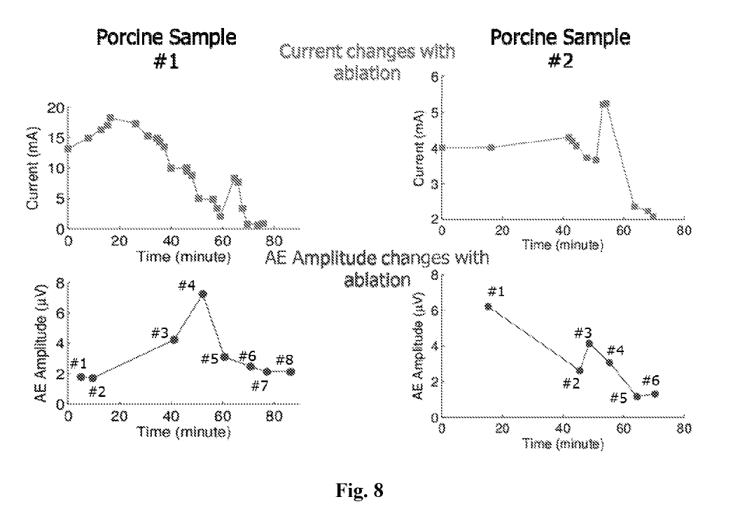

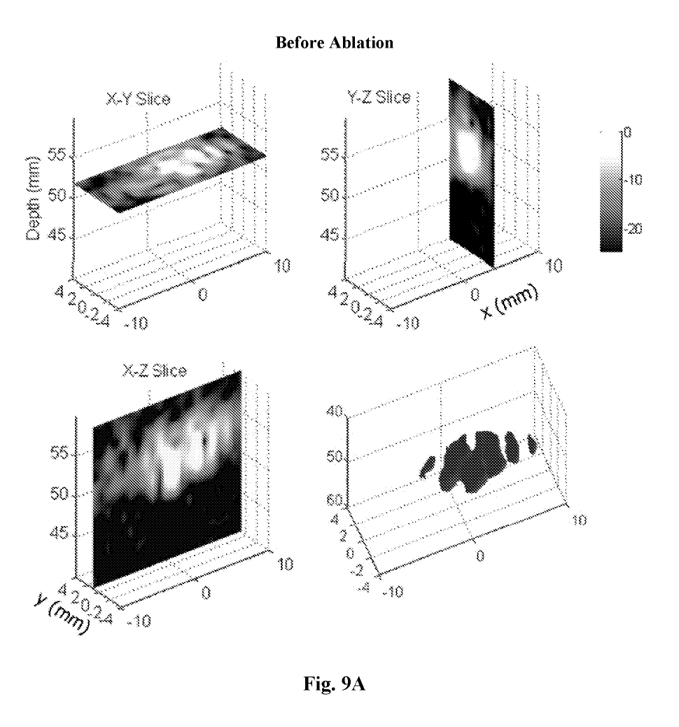

FIG. 8 is a diagram illustrating current (mA) and AE signal (μV) changes during HIFU ablation, using a comparison of two porcine samples. The figure depicts graphs that show the changes in the current and AE signal during the ablation periods for each porcine sample described in the previous figures. As seen in the UCSDI images, the AE signal initially increases during ablation, then decreases as the tissue degrades.

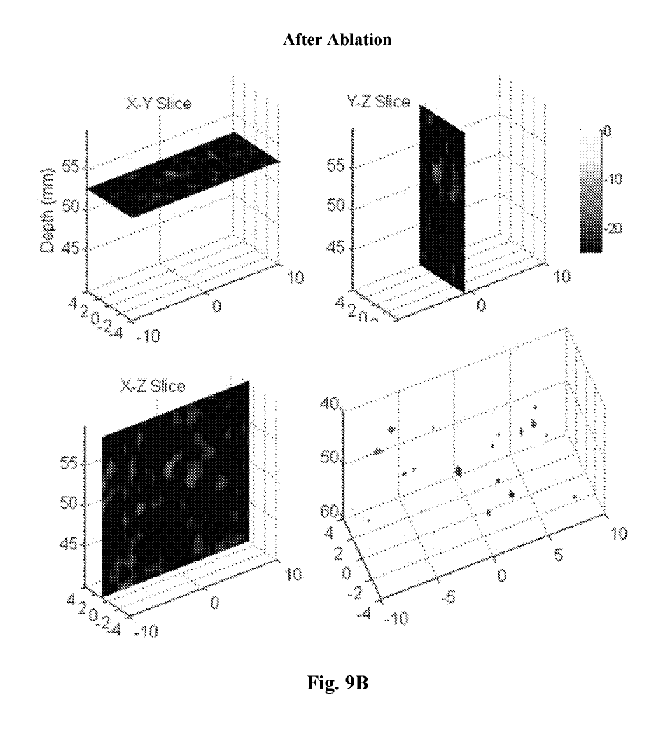

FIGS. 9A and 9B are diagrams illustrating 3D UCSDI representations (volumes) of preserved porcine sample before and after ablation, respectively. Volume UCSDI images of the preserved porcine sample before (FIG. 9A) and after (FIG. 9B) ablation (dynamic range=20 dB). FIG. 9A shows the HIFU ablation-spot corresponding to FIG. 4, frame #4; FIG. 9B shows that, by frame #8, the tissue is completely degraded and passes almost no current. There is no visible ablated region in the final image because virtually no AE signal is generated.

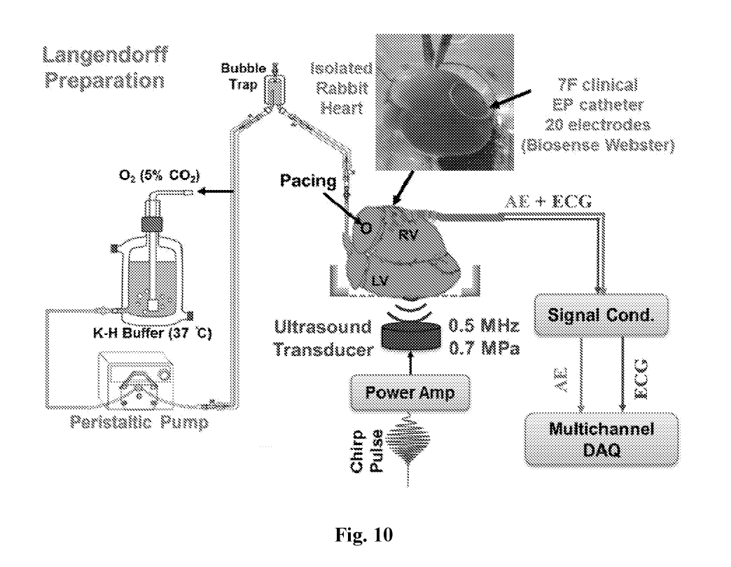

FIG. 10 is a diagram illustrating an embodiment of acoustoelectric cardiac imaging before, during, and after cryoablation. The figure shows a set-up for UCSDI in rabbit heart (acoustoelectric cardiac imaging) with cryoablation.

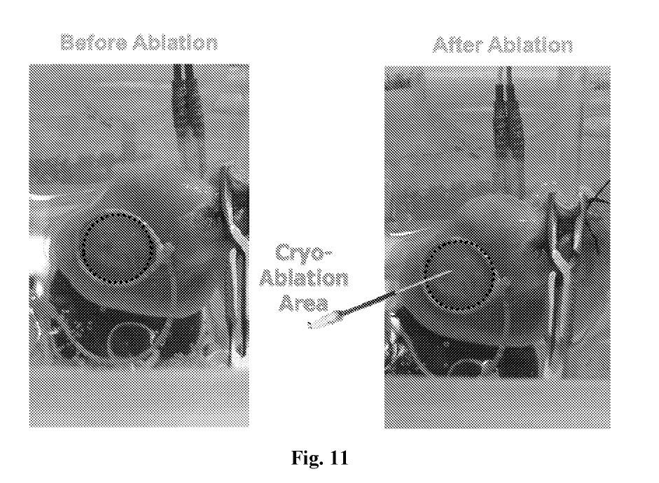

FIG. 11 are diagrams depicting images of a rabbit heart with an electrode catheter on epicardium before/after cryoablation (at site indicated on right). Acoustoelectric images and ECG were acquired at each interval.

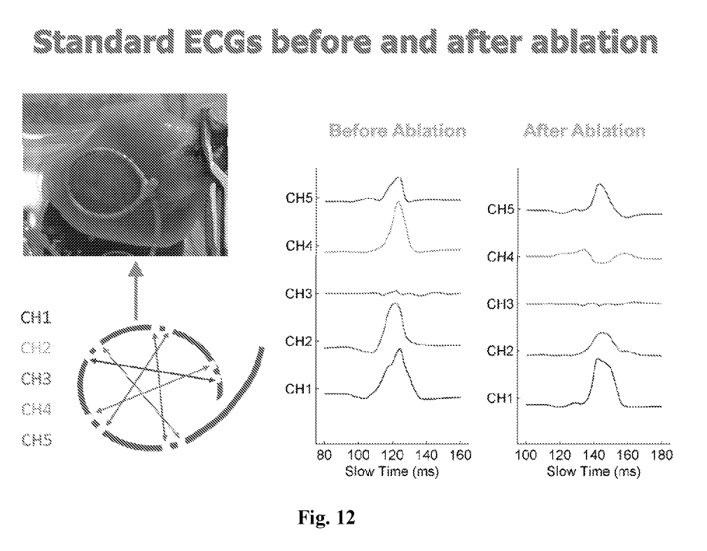

FIG. 12 is a diagram illustrating standard ECGs before and after cryoablation on 5 channels as indicated. Channel 4 (CH4) shows a dramatic change after cryoablation.

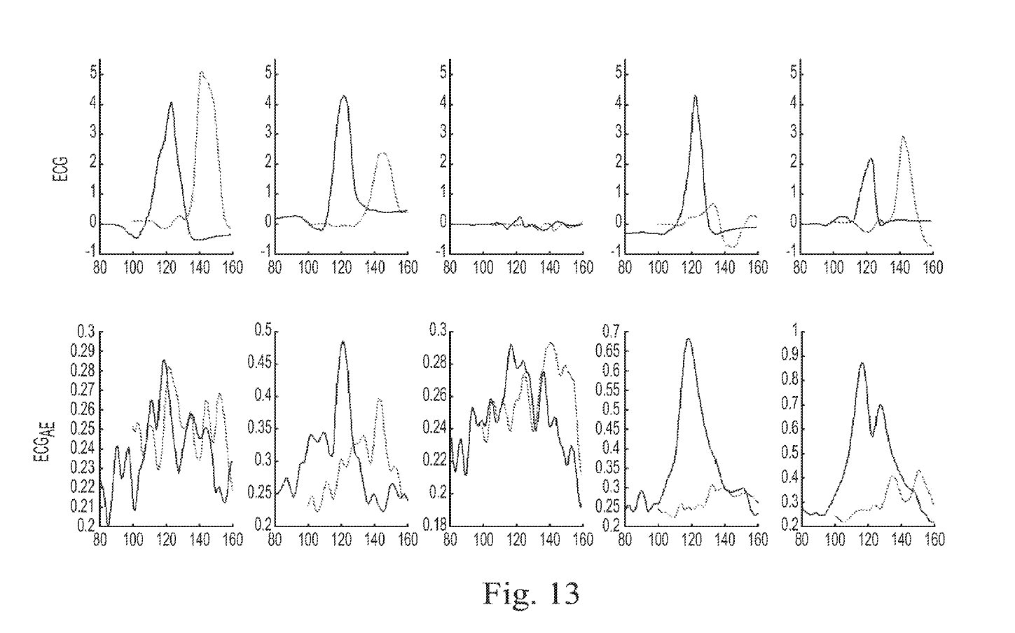

FIG. 13 is a diagram illustrating plots of standard ECG and acoustoelectric ECG at one position of US beam, before (darker lines) and after (lighter lines) cryoablation.

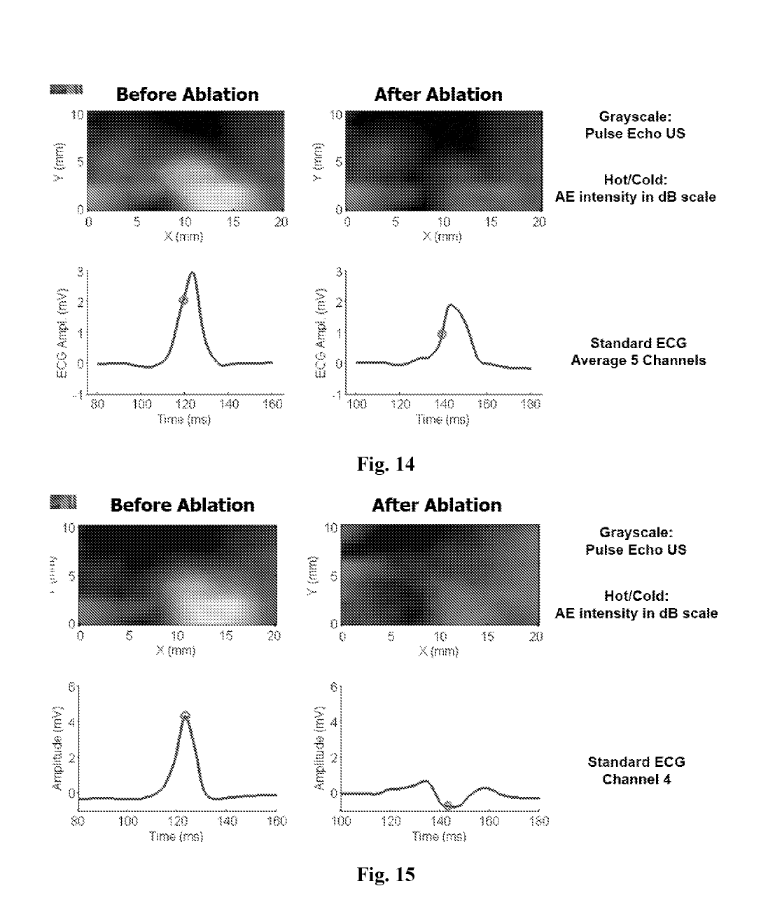

FIG. 14 is a diagram illustrating an AE image (XY slice, top row) and ECG (bottom row) before ablation (left column) and after ablation (right column). The images and ECG represent an AVERAGE of 5 channels as depicted in FIG. 12. A dramatic change in the intensity and spatial and temporal pattern is observed in the image after cryoablation.

FIG. 15 is a diagram illustrating an AE image (XY slice, top row) and ECG (bottom row) before ablation (left column) and after ablation (right column). The images and ECG represent data for just channel 4 as depicted in FIG. 12. A dramatic change in the intensity and spatial and temporal pattern is observed in the image after cryoablation.

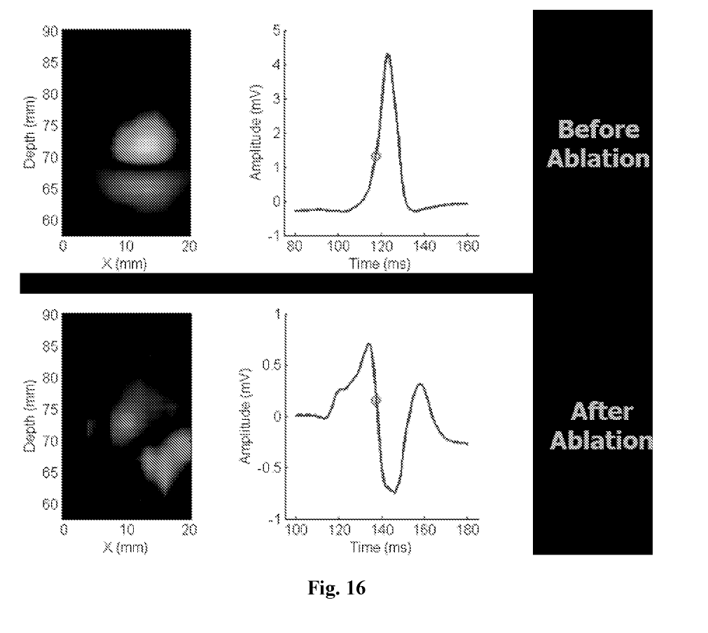

FIG. 16 is a diagram illustrating an AE B Mode color image (XZ) for Channel 4 only showing cross-section through right ventricle, before and after ablation. Color indicates direction of current field relative to the lead, whereas the intensity is related to the amplitude of the current density. Images represent a snapshot in time indicated by a red circle (in plot on right-side of images) on the ECG waveform.

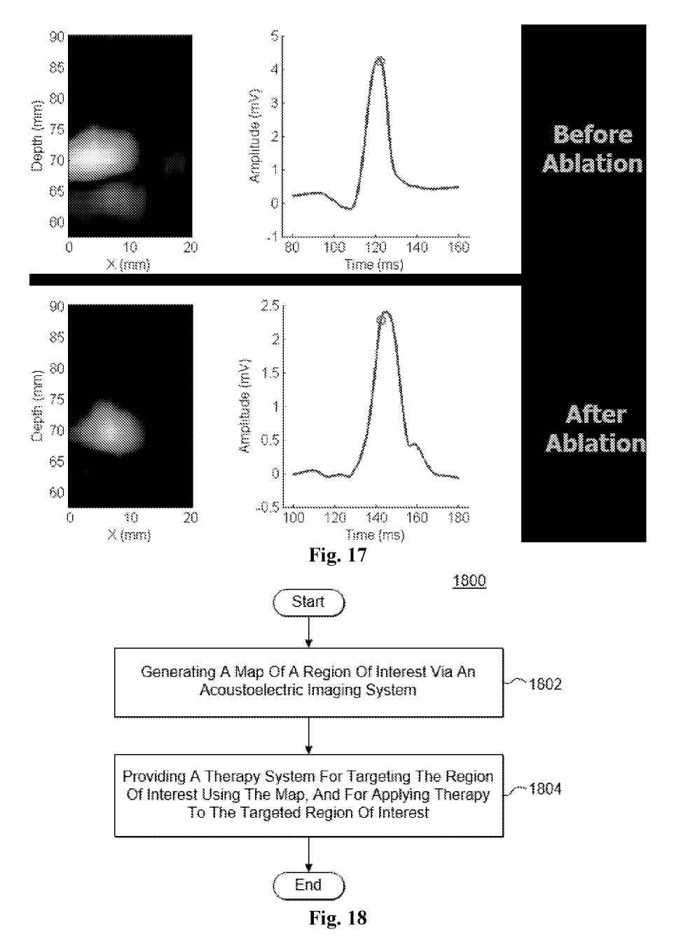

FIG. 17 is a diagram illustrating an AE B Mode color image (XZ) for Channel 2 only showing cross-section through right ventricle, before and after ablation. Color indicates direction of current field relative to the lead, whereas the intensity is related to the amplitude of the current density. Images represent a snapshot at time indicated by a red circle (in plot on right-side of images) on the ECG waveform. The pattern is similar before and after cryoablation for this channel, despite an overall increased delay typically observed during the time period of the procedure.

Although embodiments are described in this disclosure with reference to ultrasound-type therapies, other therapies such as ablation, hyperthermia, cryotherapy, cavitation, etc., may alternatively or additionally be employed and are considered to be within the spirit and scope of the present invention, and may therefore utilize the advantages of the configurations and embodiments described herein. For example, the following list of ultrasound-induced therapies may be combined with acoustoelectric imaging for guided feedback, in accordance with embodiments of the present invention:

1) Transient opening of the blood brain barrier (BBB) (e.g., to deliver drugs to the brain);

2) Sonoporation (e.g., for gene delivery);

3) Ablation (high temperature); also known as HIFU or focused ultrasound (FUS);

4) Hyperthermia (local and regional); ablation is one type with high temperatures;

5) Cavitation (high pressure); also known as histotripsy;

6) Necrosis (e.g., via cavitation or ablation);

7) Coagulation of blood vessels;

8) Sonothrombolysis (break up clots to prevent stroke);

9) Sonolysis;

10) Lithotripsy; and

11) Neuromodulation, modulation of electrical circuits (without causing damage), neural plasticity.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. An acoustoelectric image-guided therapy system comprising:
an acoustoelectric imaging system that generates a map of a region of interest; and
a therapy system that targets the region of interest using the map, and that applies therapy to the targeted region of interest.

2. The acoustoelectric image-guided therapy system of claim 1, wherein the therapy system is one of an ultrasound therapy system and an ablation therapy system.

3. (canceled)

4. The acoustoelectric image-guided therapy system of claim 1, wherein the therapy system is selected from the group consisting of ultrasound ablation, radio frequency (RF) ablation, cryoablation, hyperthermia ablation, and a combination thereof.

5. The acoustoelectric image-guided therapy system of claim 1, wherein the therapy system is selected from the group consisting of transient opening of the blood brain barrier (BBB), sonoporation, ablation, hyperthermia, cavitation, necrosis, coagulation of blood vessels, sonothrombolysis, sonolysis, lithotripsy, neuromodulation, and a combination thereof.

6. The acoustoelectric image-guided therapy system of claim 1, wherein the acoustoelectric imaging system is selected from the group consisting of pulse-echo ultrasound, ultrasound current source density imaging (UCSDI), and a combination thereof.

7. The acoustoelectric image-guided therapy system of claim 1, wherein the therapy system is configured to use integrated feedback from the acoustoelectric imaging system while targeting the region of interest.

8. The acoustoelectric image-guided therapy system of claim 7, wherein the integrated feedback is in real-time.

9. The acoustoelectric image-guided therapy system of claim 1, wherein the acoustoelectric imaging system comprises a transducer used for generating the map, and wherein the therapy system is configured to target the region of interest using the transducer.

10. The acoustoelectric image-guided therapy system of claim 1, wherein the acoustoelectric imaging system is configured to utilize passive electrical conduction to generate the map.

11. The acoustoelectric image-guided therapy system of claim 1, wherein the acoustoelectric imaging system is configured to utilize active electrical conduction to generate the map.

12. A method for applying acoustoelectric image-guided therapy, the method comprising:
generating a map of a region of interest via an acoustoelectric imaging system; and
providing a therapy system for targeting the region of interest using the map, and for applying therapy to the targeted region of interest.

13. The method of claim 12, wherein the therapy system is one of an ultrasound therapy system and an ablation therapy system.

14. (canceled)

15. The method of claim 12, wherein the therapy system is selected from the group consisting of ultrasound ablation, radio frequency (RF) ablation, cryoablation, hyperthermia ablation, and a combination thereof.

16. The method of claim 12, wherein the therapy system is selected from the group consisting of transient opening of the blood brain barrier (BBB), sonoporation, ablation, hyperthermia, cavitation, necrosis, coagulation of blood vessels, sonothrombolysis, sonolysis, lithotripsy, neuromodulation, and a combination thereof.

17. The method of claim 12, wherein the acoustoelectric imaging system is selected from the group consisting of pulse-echo ultrasound, ultrasound current source density imaging (UCSDI), and a combination thereof.

18. The method of claim 12, wherein the therapy system uses integrated feedback from the acoustoelectric imaging system while targeting the region of interest.

19. The method of claim 18, wherein the integrated feedback is in real-time.

20. The method of claim 12, wherein the acoustoelectric imaging system comprises a transducer used for generating the map, and wherein the therapy system targets the region of interest using the transducer.

21. The method of claim 12, wherein the acoustoelectric imaging system utilizes passive electrical conduction for generating the map.

22. The method of claim 12, wherein the acoustoelectric imaging system utilizes active electrical conduction for generating the map.
